(19) United States
(12) Patent Application Publication
(10) Pub. No.: US 2011/0306546 A1
(43) Pub. Date: Dec. 15, 2011

(54) COMPOSITIONS FOR INCREASING HAIR GROWTH AND DECREASING HAIR LOSS

(75) Inventors: Antonio Armani, Richmond Hill (CA); Sara Armani, Richmond Hill (CA); Charitha Seneviratne, Mississauga (CA); Reza Nazari, Richmond Hill (CA)

(73) Assignee: ALVI ARMANI GENOMICS INC., Richmond Hill (CA)

(21) Appl. No.: 13/083,203

(22) Filed: Apr. 8, 2011

Related U.S. Application Data
(60) Provisional application No. 61/353,288, filed on Jun. 10, 2010.

(51) Int. Cl. A61K 38/18; A61P 17/14; C12N 5/07

(52) U.S. Cl. 514/8.2; 435/325

(57) ABSTRACT

The invention provides compositions and methods for increasing hair growth and decreasing hair loss. In one embodiment, the compositions comprise a plurality of hair growth agents. Optionally, the hair growth agents are selected from the group consisting of: IGF-1, FGF-2, FGF-10, PDGF-AA, Wnt-3a, noggin, ephrin-A3, sonic hedgehog (SHH), BMP-6 and hypoxanthine.

### CROSS REFERENCE TO RELATED APPLICATIONS

This non-provisional application claims priority from U.S. provisional application 61/353,288 filed on Jun. 10, 2010, which is incorporated herein by reference in its entirety.

### FIELD

This application relates to compositions and methods for increasing hair growth and decreasing hair loss.

### BACKGROUND

Hair loss affects millions of people, including over 40% of men over the age of 30. Numerous factors can cause hair loss, including genetic predisposition, autoimmune reactions, scarring, disease and infection. Hair loss can ultimately lead to complete baldness.

Alopecia is a medical condition in which hair is lost from an area of the body. One symptom of alopecia is hair follicle miniaturization (described below). Alopecia includes both androgenetic alopecia, also known as male pattern baldness, and alopecia areata, which is thought to be an autoimmune disorder.

Normally, a hair follicle cycles through phases including the anagen (growth) phase, the catagen (transition) phase and the telogen (resting or quiescent) phase. In the miniaturization process, the hair follicle enters a prolonged lag phase following the telogen stage. With successive anagen cycles, the follicles become smaller, leading to shorter, finer hair. The miniaturized follicle eventually produces a tiny hair shaft that is cosmetically insignificant. Ultimately, the follicle can stop producing a hair shaft altogether and the area of hair loss can become completely devoid of hair.

Several methods for treating hair loss are available, including drugs such as topical minoxidil and orally-delivered propecia. However, these treatments have achieved limited success in restoring natural hair growth and are only effective while the drugs are being taken.

There remains a need for novel compositions and methods for treating and preventing hair loss.

### SUMMARY OF THE DISCLOSURE

The application relates to a composition comprising a plurality of hair growth agents. The application also relates to the use of the composition for increasing hair growth and/or decreasing hair loss.

In one embodiment of the invention, the invention provides a composition comprising at least four hair growth agents selected from group consisting of: IGF-1, FGF-2, FGF-10, PDGF-AA, Wnt-3a, noggin, ephrin-A3, SHH, BMP-6 and hypoxanthine. In another embodiment, the invention provides a composition comprising at least five, six, seven, eight, nine or ten hair growth agents selected from group consisting of: IGF-1, FGF-2, FGF-10, PDGF-AA, Wnt-3a, noggin, ephrin-A3, SHH, BMP-6 and hypoxanthine.

Optionally, the composition comprises at least four hair growth agents selected from group consisting of: 1 ng/ml to 100 μg/ml, optionally 10 ng/ml to 30 ng/ml, IGF-1; 1 ng/ml to 100 μg/ml, optionally 10 ng/ml to 30 ng/ml, FGF-2; 1 ng/ml to 100 μg/ml, optionally 10 ng/ml to 30 ng/ml FGF-10; 1 ng/ml to 100 μg/ml, optionally 10 ng/ml to 30 ng/ml, PDGF-AA; 1 ng/ml to 100 μg/ml, optionally 10 ng/ml to 30 ng/ml, Wnt-3a; 1 ng/ml to 100 μg/ml, optionally 10 ng/ml to 30 ng/ml, noggin; 1 ng to 100 μg/ml, optionally 10 ng/ml to 30 ng/ml ephrin-A3; 1 ng to 100 μg/ml, optionally 10 ng/ml to 30 ng/ml SHH; 1 ng/ml to 100 μg/ml, optionally 10 ng/ml to 30 ng/ml, BMP-6; and 1 nM to 1 mM, optionally 1 μM to 3 μM, hypoxanthine. In another embodiment, the composition comprises at least four hair growth agents selected from the group consisting of: 15-25 ng/ml IGF-1, 15-25 ng/ml FGF-2, 15-25 ng/ml FGF-10, 15-25 ng/ml PDGF-AA, 15-25 ng/ml Wnt-3a, 15-25 ng/ml noggin, 15-25 ng/ml ephrin-A3, 15-25 ng/ml SHH, 15-25 ng/ml BMP-6, and 1.5-2.5 μM hypoxanthine. In yet another embodiment, the composition comprises at least four hair growth agents selected from the group consisting of: 20 ng/ml IGF-1; 20 ng/ml FGF-2; 20 ng/ml FGF-10, 20 ng/ml PDGF-AA; 20 ng/ml Wnt-3a; 20 ng/ml noggin; 20 ng/ml ephrin-A3; 20 ng/ml SHH; 20 ng/ml BMP-6; and 2 μM hypoxanthine.

In another embodiment of the invention, the composition comprises IGF-1, FGF-2, PDGF-AA, Wnt-3a, noggin, BMP-6 and hypoxanthine. In another embodiment, the composition comprises 1 ng/ml to 100 μg/ml, optionally 10 ng/ml to 30 ng/ml, IGF-1; 1 ng/ml to 100 μg/ml, optionally 10 ng/ml to 30 ng/ml, FGF-2; 1 ng/ml to 100 μg/ml, optionally 10 ng/ml to 30 ng/ml, PDGF-AA; 1 ng/ml to 100 μg/ml, optionally 10 ng/ml to 30 ng/ml, Wnt-3a; 1 ng/ml to 100 μg/ml, optionally 10 ng/ml to 30 ng/ml, noggin; 1 ng/ml to 100 μg/ml, optionally 10 ng/ml to 30 ng/ml, BMP-6; and 1 nM to 1 mM, optionally 1 μM to 3 μM, hypoxanthine. In another embodiment, the composition comprises 15-25 ng/ml IGF-1, 15-25 ng/ml FGF-2, 15-25 ng/ml PDGF-AA, 15-25 ng/ml Wnt-3a, 15-25 ng/ml noggin, 15-25 ng/ml BMP-6 and 1.5-2.5 μM hypoxanthine. In yet another embodiment, the composition comprises 20 ng/ml IGF-1; 20 ng/ml FGF-2; 20 ng/ml PDGF-AA; 20 ng/ml Wnt-3a; 20 ng/ml noggin; 20 ng/ml BMP-6; and 2 μM hypoxanthine. In a further embodiment, the composition consists essentially of IGF-1, FGF-2, PDGF-AA, Wnt-3a, noggin, BMP-6 and hypoxanthine.

In another embodiment of the invention, the composition comprises IGF-1, FGF-2, FGF-10, PDGF-AA, Wnt-3a, noggin, ephrin-A3, SHH, BMP-6 and hypoxanthine. Optionally, the composition comprises 1 ng/ml to 100 μg/ml, optionally 10 ng/ml to 30 ng/ml, IGF-1; 1 ng/ml to 100 μg/ml, optionally 10 ng/ml to 30 ng/ml, FGF-2; 1 ng/ml to 100 μg/ml, optionally 10 ng/ml to 30 ng/ml FGF-10; 1 ng/ml to 100 μg/ml, optionally 10 ng/ml to 30 ng/ml, PDGF-AA; 1 ng/ml to 100 μg/ml, optionally 10 ng/ml to 30 ng/ml, Wnt-3a; 1 ng/ml to 100 μg/ml, optionally 10 ng/ml to 30 ng/ml, noggin; 1 ng/ml to 100 μg/ml, optionally 10 ng/ml to 30 ng/ml ephrin-A3; 1 ng/ml to 100 μg/ml, optionally 10 ng/ml to 30 ng/ml SHH; 1 ng/ml to 100 μg/ml, optionally 10 ng/ml to 30 ng/ml, BMP-6; and 1 nM to 1 mM, optionally 1 μM to 3 μM, hypoxanthine. In another embodiment, the composition comprises 15-25 ng/ml IGF-1, 15-25 ng/ml FGF-2, 15-25 ng/ml FGF-10, 15-25 ng/ml PDGF-AA, 15-25 ng/ml Wnt-3a, 15-25 ng/ml noggin, 15-25 ng/ml ephrin-A3, 15-25 ng/ml SHH, 15-25 ng/ml BMP-6, and 1.5-2.5 μM hypoxanthine. In yet another embodiment, the composition comprises 20 ng/ml IGF-1; 20 ng/ml FGF-2; 20 ng/ml FGF-10, 20 ng/ml PDGF-AA; 20 ng/ml Wnt-3a; 20 ng/ml noggin; 20 ng/ml ephrin-A3; 20 ng/ml SHH; 20 ng/ml BMP-6; and 2 μM hypoxanthine. In a further embodiment, the composition consists essentially of IGF-1, FGF-2, FGF-10, PDGF-AA, Wnt-3a, noggin, ephrin-A3, SHH, BMP-6 and hypoxanthine.

In another embodiment, the invention relates to the use of the compositions of the invention to increase hair growth. Optionally, the hair is a hair follicle in vitro or in vivo. Optionally, the compositions are for use in an amount of 1-3 ml per day, optionally 2 ml per day. In another embodiment, each of IGF-1, FGF-2, FGF-10, PDGF-AA, Wnt-3a, noggin, ephrin-A3, SHH and BMP-6 are for use in an amount of 20 to 80 ng/day, optionally 40 ng/day, and hypoxanthine is for use in an amount of 2 to 8 nmoles/day, optionally 4 nmoles/day.

The invention also relates to the use of the compositions of the invention to decrease hair loss. Optionally, the compositions are for use in an amount of 1-3 ml per day, optionally 2 ml per day. Optionally, IGF-1, FGF-2, FGF-10, PDGF-AA, Wnt-3a, noggin, ephrin-A3, SHH and BMP-6 are for use in an amount of optionally 20 to 80 ng/day, 30 to 50 ng/day or 40 ng/day, and hypoxanthine is for use in an amount of optionally 2 to 8 nmoles/day, 3 to 5 nmoles/day or 4 nmoles/day.

The invention also relates to the use of the compositions of the invention to increase the viability of a hair follicle cell, in vitro or in vivo. Optionally, the hair follicle cell is selected from the group consisting of: epidermal matrix cells, dermal papilla cells, dermal sheath cells and outer root sheath cells.

The invention further relates to a method of increasing the viability of a hair follicle cell, wherein the method comprises contacting the hair follicle cell with the composition of claim 1. In one embodiment, the hair follicle cell is a cell selected from the group consisting of: epidermal matrix cells, dermal papilla cells, dermal sheath cells and outer root sheath cells.

In a further embodiment, the invention relates to a method of increasing hair growth and/or decreasing hair loss in a subject in need thereof, wherein the method comprises administering the compositions of the invention to the subject. Optionally, the composition administered topically. Optionally, the composition is administered twice daily. In one embodiment, optionally 0.25 ml to 10 ml, 0.5 to 5 ml, 1 to 3 ml, or 2 ml of the composition is administered per day. In another embodiment, optionally 20 to 80 ng, 30 to 50 ng or 40 ng of each of IGF-1, FGF-2, FGF-10, PDGF-AA, Wnt-3a, noggin, ephrin-A3, SHH and BMP-6 is administered daily and optionally 2 to 8 nmoles, 3 to 5 nmoles or 4 nmoles of hypoxanthine is administered daily.

In one specific embodiment, the composition is administered to the scalp of the subject, optionally to a thinning hair patch or a bald patch on the scalp.

In yet another embodiment, the invention relates to a method of preparing a composition useful increasing hair growth or decreasing hair loss, the method comprising:
providing a composition comprising at least four hair growth agents selected from group consisting of: IGF-1, FGF-2, FGF-10, PDGF-AA, Wnt-3a, noggin, ephrin-A3, SHH, BMP-6 and hypoxanthine.

Optionally, the method further comprises administering the composition to a subject to increase hair growth or decrease hair loss in the subject.

### BRIEF DESCRIPTION OF THE DRAWINGS

Embodiments of the invention will be shown in relation to the drawings in which the following is shown:

FIG. 1. Hair follicle (HF) explant growth assays for individual growth factors, hypoxanthine, and growth cocktails GF7 and GF10. Each experimental point represents the mean±SEM of 6-8 independent experiments where an increase in HF growth as a % of the untreated control is expressed.

FIG. 2. Dermal papilla (DP) cell viability when treated with growth cocktail GF10 at various concentrations.

FIG. 3. Bald (hair loss) areas/zones of the scalp.

FIG. 4. Growth factor profiles for individual patients. Hair follicles extracted from individual patients were treated in vitro with different growth factors. Growth was measured after 7-8 days and expressed as % of the control (no growth factors) for each growth factor.

FIG. 5. Bald area 1 of patient 1 before and after treatment with growth cocktail GF10.

FIG. 6. Bald area 3 of patient 1 before and after treatment with growth cocktail GF10.

FIG. 7. Bald area 2 of patient 2 before and after treatment with growth cocktail GF10.

FIG. 8. Bald area 3 of patient 2 before and after treatment with growth cocktail GF10.

FIG. 9. Bald area 1 of patient 3 before and after treatment with growth cocktail GF10.

FIG. 10. Photographs of a subject before and after 3 months of treatment with the GF10 composition.

### DETAILED DESCRIPTION

The application relates to a composition comprising a plurality of hair growth agents. The application also relates to the use of the composition for increasing hair growth and/or decreasing hair loss.

The term “hair growth agent” refers to any cellular protein, nucleic acid, polysaccharide or lipid that is associated with stimulating, maintaining, or increasing the growth of a hair, for example, increasing the length or diameter of the hair shaft.

A hair growth agent, for example, may stimulate a hair follicle or hair follicle cells. In one embodiment of the invention, a “hair growth agent” is a protein, optionally a cellular growth factor. In another embodiment of the invention, a “hair growth agent” is hypoxanthine, a naturally occurring purine derivative. Hypoxanthine is a precursor to nucleotide guanine found in DNA and, without wishing to be bound by theory, enhances the stem cell reservoir in the hair follicle bulge (U.S. Pat. No. 7,655,465).

The term “cellular growth factor” refers to a naturally occurring substance capable of stimulating cellular growth, proliferation and differentiation. Examples of cellular growth factors that play a role in hair follicle development include, but are not limited to: IGF-1 (insulin-like growth factors-1), FGF-2 (fibroblast growth factor-2), FGF-10, PDGF-AA (platelet-derived growth factor-AA), Wnt-3a, Noggin, Ephrin-A3, SHH (sonic hedgehog) and BMP-6 (bone morphogenesis protein-6). Without wishing to be bound by theory, a brief description of certain properties of the various growth factors follows:

The insulin-like growth factors (IGFs) comprise a family of peptides that play important roles in mammalian growth and development. IGF-1 has been suggested to stimulate hair follicle growth in a dose-dependant manner (Philpott et al., 1994).

The involvement of FGF-2 in hair follicle and hair follicle cell growth has been studied. FGF-2 is present adjacent to the proliferative zone of the mature follicle. The controlled release of FGF-2 has been suggested to positively affect hair growth cycles in mice (Ozaki and Tabata, 2003).

FGF-10, also known as keratinocyte growth factor (KGF2), is found in dermal papilla fibroblasts while its receptor is found in the neighboring keratinocytes (Katsuoka et al., 1987). Human recombinant FGF-10 was suggested to stimulate the growth of human hair follicle explants derived from both normal scalps and alopecia scalps (Jang, 2005).

PDGF-AA is a member of the platelet derived growth factor family. Mice lacking PDGF-AA have small dermal papilla cells, dermal sheath abnormalities and thin hair compared to their wild type siblings (Karlsson et al., 1999).

Wnt-3a has been studied in hair follicle differentiation. Subcutaneous administration of human recombinant Wnt-3a to mice was suggested to result in robust induction of hair follicle neogenesis compared to untreated controls (Morrell et al., 2008). It has also been suggested that the presence of Wnt-3a in the cell culture medium is necessary in order for cultured dermal papilla cells to maintain their hair inducing activity (Kishimoto et al., 2000).

The secreted polypeptide noggin binds and inactivates members of the transforming growth factor-beta (TGF-beta) superfamily signaling proteins. Mice lacking noggin have a lower number of hair follicles compared to their normal littermates. Further, ectopic expression of noggin in chick or mouse embryonic skin was believed to cause enlargement of ectopic follicles in one study (Noramly and Morgan, 1998; Bochkarev et al., 1999).

Ephrin-A3 is a member of the Ephrin family and binds to the Eph receptor. It is implicated in the development of the nervous system and erythropoiesis (Holder and Klein et al., 1999). Cultured dermal papilla cells from subjects with androgenic alopecia show a 100-fold decrease in Ephrin-A3 expression by microarrays compared to that of normal controls.

Sonic hedgehog (SHH) is believed to play a role in hair follicle development. In mice lacking SHH, hair follicle formation is initiated and the dermal condensate is formed, but mature hair follicles fail to develop (St-Jacques et al., 1998; Chiang et al., 1998; Karlsson et al., 1999).

Bone morphogenetic factors (BMPs) are TGF-beta family members and are believed to be important in hair follicle differentiation (O'Shaughnessy et al., 2004) and act through their cognate receptor, BMPR1a. This receptor is in all different hair bulb cell populations but especially prominent in DP cells (Rendl et al., 2005). BMP-6 is expressed in DP cells and is most potent in maintaining the DP cell molecular signature in vitro and hair follicle inducibility in vivo (Rendl et al., 2008).

In the present application, the term “hair follicle” refers to a tube-like tissue having an opening in which a hair shaft develops. The hair follicle is typically located in the epidermis. The hair follicle may be optionally isolated (extracted) from the epidermis using known techniques. A hair follicle typically includes the following structures: papilla, matrix, root sheath, sebaceous gland and hair fiber (also known as a hair shaft). In a subject with hair loss, the hair shaft may be undergoing the miniaturization process or may not present at all, depending on the extent of the alopecia.

The term “hair follicle cell” refers to a cell that is present in, or derived from, a hair follicle. Hair follicle cells include, but are not limited to, dermal papilla (DP) cells, dermal sheath (DS) cells, outer root sheath (ORS) cells and epidermal matrix (EM) cells.

The term “increases hair growth” includes, but is not limited to, activity that increases the number of hairs on a mammal, maintains the number of hairs in a given area of scalp on a mammal that would otherwise experience net hair loss, grows hair on a mammal, re-grows hair on a mammal, increases the length or thickness (diameter) of a hair shaft on a mammal, improves the health of hair on a mammal, treats baldness (for example, male pattern baldness, female pattern baldness, genetic alopecia) and/or increases hair follicle density. The term “increasing hair growth” includes activity that stimulates growth of a single hair in a follicle or growth of a group of hairs in hair follicles in specified area of epidermis. Increasing hair growth optionally occurs, for example, by increasing the number of hairs present in an area of epidermis of a mammal or maintaining the number of hairs present in an area of epidermis of a mammal that would otherwise experience net hair loss (optionally measured per square cm). Increasing hair growth optionally causes growth of a new hair in a follicle (e.g. after a hair has fallen out) or increases rate of growth of an existing hair (length and/or width) of a hair in a follicle on a mammal. Increasing hair growth optionally increases hair length. Increasing hair growth prevents (reduces) and/or treats baldness and/or balding. It optionally has other effects such as increasing hair follicle density in an area and/or the appearance of thickness of hair in an area. Increasing hair growth optionally also improves the health of hair and hair follicles on a mammal. Typically the increase in the foregoing parameters that are quantifiable will be at least: 5%, 10%, 20%, 50%, 100% or 150% compared to untreated hair follicles (or epidermis) that do not experience the present methods and compositions that increase hair growth. These percentage increases are optionally measured in a single hair or single hair follicle (e.g. rate of increased growth, increase in length or thickness per day) or in a plurality of hairs or hair follicles in a specified area (e.g. increase in number of hairs per square cm or in length of hairs growing per square cm).

The term “increasing hair growth” optionally refers to increasing the viability of hair follicles in vivo or in vitro. The term “increasing hair growth” also optionally refers to increasing the viability of an isolated hair follicle, i.e. an isolated hair follicle in culture (in vitro). Increasing the viability of hair follicles in vitro can be measured through a hair follicle explant growth assay, a hair follicle explant viability assay or any other method known in the art. Typically the increase in the foregoing parameters will be at least: 5%, 10%, 20%, 50%, 100% or 150% compared to untreated hair follicles that do not experience the present methods and compositions that increase hair growth.

The term “decreases hair loss” includes, but is not limited to, activity that maintains the number of hairs or hair follicles on a mammal that would otherwise experience net hair loss (optionally measured as the number of hairs or hair follicles measured per square cm), reduces the rate of balding and/or reduces the rate of hair follicle miniaturization. Decreasing hair loss optionally decreases the rate of hair loss, hair follicle loss and/or hair follicle miniaturization by at least 5%, 10%, 20%, 50%, 100% or 150% compared to untreated hair follicles (or epidermis) that do not experience the present methods and compositions that decrease hair loss. These percentage increases are optionally measured in a single hair or single hair follicle or in a plurality of hairs or hair follicles in a specified area.

The term “decreases hair loss” includes, but is not limited to, activity that maintains the number of hairs or hair follicles on a mammal that would otherwise experience net hair loss (optionally measured as the number of hairs or hair follicles measured per square cm), reduces the rate of balding and/or reduces the rate of hair follicle miniaturization. Decreasing hair loss optionally decreases the rate of hair loss, hair follicle loss and/or hair follicle miniaturization by at least 5%, 10%, 20%, 50%, 100% or 150% compared to untreated hair follicles (or epidermis) that do not experience the present methods and compositions that decrease hair loss. These percentage increases are optionally measured in a single hair or single hair follicle or in a plurality of hairs or hair follicles in a specified area.

The term “increases cell viability” refers to increasing the viability of cells, whether in vivo or in vitro. The term “increases isolated cell viability” refers to increasing the viability of isolated cells in culture (in vitro). The term can refer to increasing the growth of one or more hair follicle cells such as dermal papilla cells, outer root sheath cells, epidermal stem cells, dermal sheath cells or epidermal matrix cells. In one example, cell viability is determined by incubating cells with methanethiosulfonate (MTS) reagents and measuring optical density (OD) 490 nm spectrophotometrically. Optionally, increased cell viability is indicated by an increase in the percent survival of treated cells versus non-treated cells. Typically, the increase in cell viability will be quantifiable, for example, 110%, 120%, 150%, 200% or 500% viability compared to a control.

The term “increases hair follicle viability” refers to increasing the viability of hair follicles, whether in vivo or in vitro. The term “increases isolated hair follicle viability” refers to increasing the viability of isolated hair follicles in culture (in vitro). Optionally, increased hair follicle viability is indicated by an increase in the percent survival of treated hair follicles versus non-treated hair follicles. Typically, the increase in hair follicle viability will be quantifiable, for example, 110%, 120%, 150%, 200% or 500% viability compared to a control. Hair follicle viability is assessed by any method known in the art to quantify hair follicle viability, optionally a hair follicle explant assay.

In one aspect of the invention, the composition comprises, consists essentially of or consists of includes all or a sub-set of hair growth agents IGF-1, FGF-2, FGF-10, PDGF-AA, HGF, SCF, Wnt-3a, Noggin, BMP-6, hypoxanthine, SHH, and Ephrin A3. In one embodiment, the composition comprises, consists essentially of or consists of the following 7 hair growth agents: IGF-1, FGF-2, PDGF-AA, Wnt-3a, noggin, BMP-6 and hypoxanthine. In another embodiment, the composition comprises, consists essentially of or consists of the following 10 hair growth agents: IGF-1, FGF-2, FGF-10, PDGF-AA, Wnt-3a, noggin, ephrin-A3, SHH, BMP-6 and hypoxanthine. Each of the cellular hair growth factors (IGF-1, FGF-2, FGF-10, PDGF-AA, Wnt-3a, noggin, ephrin-A3, SHH, BMP-6) may be present in the composition in an amount of 1 ng to 100 μg/ml, optionally 10 ng/ml to 30 ng/ml, optionally 20 ng/ml. Hypoxanthine may be present in the composition in an amount of 1 nM to 1 mM, optionally 1 μM to 3 μM, optionally 2 μM. Optionally, the composition includes plant extracts, fractions thereof, and stem cell mobilizing factors from plant extracts or fractions.

The compositions of the invention optionally contain between 0.01% to 100% by weight of the hair growth agents. The dosage of the compositions varies according to the specific form of the external application, age and the type and degree of hair loss. Optionally, the composition is administered to a subject's head in an amount of 1 to 3 ml per day, optionally 2 ml/day.

In one aspect of the invention, the invention relates to the use of the present compositions to increase hair growth or decrease hair loss. The invention further relates to the use of the present compositions to generate new hair on a subject. In one aspect of the invention, a new hair is generated from a pre-existing follicle. In another aspect of the invention, a follicle giving rise to a new hair is generated. The generation of new hair may comprise increasing the density of individual hairs and/or hair follicles within a specified area of a patient's scalp. Optionally, hair density is increased by 5%, 10%, 20%, 50% or more than 100%. In one embodiment of the invention, the present compositions are topically applied to a subject for use in generating new hair.

The invention further relates the use of the present compositions to thicken a hair shaft on a subject. Optionally, the diameter of a thickened hair shaft is increased by 5%, 10%, 20%, 50% or more than 100% following treatment with a composition of the invention. Optionally, the diameter of a thickened hair shaft is increased by at least 10-100 μm, optionally 20-50 μm.

The invention further relates the use of the present compositions to increase the rate of hair growth on a subject. Optionally, the rate is increased by 5%, 10%, 20%, 50% or more than 100% following treatment with a composition of the invention. The invention also relates to the use of the present compositions to increase the longitudinal hair growth of a subject. Optionally, longitudinal hair growth is increased by 5%, 10%, 20%, 50% or more than 100% following treatment with a composition of the invention.

The invention also relates to the use of the present compositions to increase the viability of hair follicles in vitro or in vivo.

The invention also relates to the use of the present compositions to increase the viability of hair follicle cells, for example, outer root sheath cells, epidermal stem cells, dermal papilla cells, dermal sheath cells and epidermal matrix cells.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Suitable vehicles are described, for example, in Remington's Pharmaceutical Sciences (2003-20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which optionally further contain antioxidants, buffers, bacteriostats and solutes that render the compositions substantially compatible with the tissues or the blood of an intended recipient. Other components that are optionally present in such compositions include, for example, water, surfactants (such as Tween™), alcohols, polyols, glycerin and vegetable oils. Extemporaneous injection solutions and suspensions may be prepared from sterile powders, granules, tablets, or concentrated solutions or suspensions. The composition can be supplied, for example but not by way of limitation, as a lyophilized powder which is reconstituted with sterile water or saline prior to administration to the subject.

Suitable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound(s), together with a suitable amount of carrier so as to provide the form for direct administration to the subject.

Optionally, the composition of the invention is formulated in a suitable dermal penetration carrier or pharmaceutically acceptable carrier. Optionally, the carrier is a cosmetic carrier. The carrier may contain antioxidants, vitamins, preservatives, anti-microbials, colorants, moisturizers, thickeners and preservatives that do not interfere with the desired effects of the present invention

In one embodiment, the composition is administered to the patient's epidermis, typically the scalp. Optionally, the composition is administered to a specific area of the scalp. In one embodiment of the invention, the specific area of the scalp includes a thinning hair patch, a bald patch, or an area with no balding (ie normal hair growth).

In one embodiment, the composition of the invention is a topical composition typically applied to the scalp or skin by spraying or coating. Optionally, the compositions for external dermal applications are formulated as liquids, milky lotions, gels, creams, aerosols, sprays, powders or rinses. There are no limitations to the method by which the compositions can be applied. For example, 1 to 5 ml of the compositions could be applied to scalp or skin surface areas 1 to 3 times per day.

In one embodiment of the invention, the composition is administered to the scalp by injection. In another embodiment, the composition is administered by surgical implantation.

The administration of the composition into the scalp may be intrafollicular (in the hair follicle) or interfollicular (between hair follicles). In one embodiment of the invention, the composition is injected into hair follicles undergoing the miniaturization process. In another embodiment, the composition is injected into miniaturized hair follicles. In another embodiment, the composition is administered to a bald area containing no hair or hair follicles. In another embodiment of the invention, the composition is injected into incisions in the scalp.

The injection is performed with any type of syringes, such as insulin syringes, Hamilton syringes, etc., or micropipettes.

According to one embodiment of the invention, 1 microliter to 1000 microliters, optimally 10 to 50 microliters of the personalized composition is injected.

In one aspect of the invention, the compositions are used for treating hair loss or baldness. Optionally, the compositions are also used for preventing or reducing hair loss or baldness (e.g. stopping or slowing hair loss progression). The compositions are therefore useful by themselves or as additives to products such as shampoo, conditioner, mousses, gels or creams as well as other cosmetics and drugs (typically over the counter drugs). These products are topically administered according to methods described herein.

In one aspect of the invention, the composition is tailored for a specific person. In another aspect, the composition is tailored for a specific group of individuals sharing one or more common characteristics (for example, gender, age, ethnicity, pattern of hair loss or type of hair loss). In another aspect, the composition is tailored for a specific area of hair loss. In yet another aspect, the composition is tailored both for a specific person and for a specific area of hair loss. Different compositions may be administered to different scalp areas of the same individual.

The dosage of the composition may vary according to the specific form of the external application, age and the type and degree of hair loss. Optionally, the compositions of the invention are administered to subjects with hair loss as classified by the Norwood scale of hair loss as class 2 (mild hair loss), class 3 (mild to moderate hair loss), class 4 (moderate hair loss), class 5 (moderate to large hair loss), class 6 (large hair loss) or class 7 (complete hair loss). Optionally, the compositions of the invention are administered to subjects with no hair loss (class 1) in order to prevent future hair loss.

In another aspect of the invention, the composition is used conjunction with hair transplant surgery. Optionally, the composition is administered to a patient prior to surgery, during surgery, or following surgery. The invention therefore relates to a method of transplanting hair in a subject by implanting a hair follicle in the subject and contacting the hair follicle with a composition described here. The hair follicle of the subject can be contacted with the composition prior to, during, or after transplantation. The follicle transplant is typically made onto a human scalp and the compositions are optionally used for at least one week, four weeks or at least 52 weeks.

In another aspect, the composition is used in conjunction with the process of follicular cell implantation. In follicular cell implantation, hair follicle cells (optionally, DP, DS, ORS and/or EM cells, or any combination thereof) are implanted in a patient's scalp, either into an existing hair follicle or interfollicularly. Through this method, the existing hair follicle is rejuvenated or a new follicle is generated. In one aspect, the composition is implanted with the hair follicle cells. In another aspect, the composition is mixed with the hair follicle cells prior to implantation. In another aspect of the invention, the composition is applied to the scalp or to an individual hair follicle in the scalp, prior to or following cell implantation.

In another embodiment of the invention, the composition is used to promote the viability of cells derived from hair follicles. Cells derived from hair follicles include, but are not limited to, dermal papilla cells, outer root sheath cells, dermal sheath cells and epidermal matrix cells. In one aspect of the invention, the composition is added to cell culture medium to increase the viability of hair follicle cells in vitro.

In another embodiment of the invention, the composition is used to promote the viability of explant hair follicles in vitro. In another aspect, the composition is used to increase the length of explant hair follicles in vitro. The invention therefore relates to a method of increasing the length or viability of hair follicles in vitro by contacting the hair follicle with a composition described herein. Optionally, the invention relates to a method of increasing the length or viability of hair follicles in vitro by maintaining the hair follicles in media comprising a composition described herein.

### EXAMPLES

Embodiments of the present invention will be illustrated in a non-limiting way by reference to the examples below.

### Example 1

### Assessing the Effects of a Growth Promoting Composition

### Hair Follicles

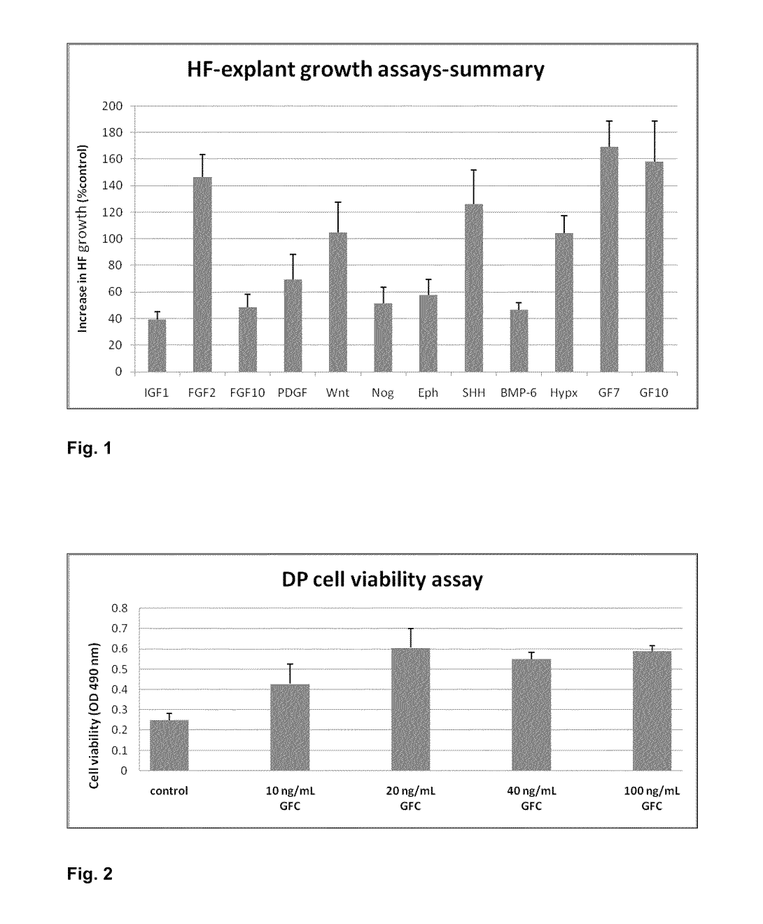

Hair follicles (HF) were extracted from the occipital area of 6-8 individuals with hair loss. The HFs were processed to equivalent lengths and cultured in William's E medium supplemented with 100 units/ml penicillin G, 100 μg/ml streptomycin, 0.25 μg/ml amphotericin B, 2 μM glutamine, 1 ng/ml hydrocortisone; basic medium or control medium or basic medium supplemented with 20 ng/ml of each of IGF-1, FGF-2, PDGF, Wnt-3A (Wnt), Noggin (Nog), Ephrin A3 (Eph), SHH and BMP-6 as well as hypoxanthine (Hyx) at 2 μM final concentration. The “GF10 cocktail” or “GF10” includes all nine growth factors and hypoxanthine while the “GF7 cocktail” or “GF7” contains six growth factors (all growth factors of GF10 except for FGF-10, Eph and SHH) and hypoxanthine. Growth promotion was measured as elongation of the HF at the end of 7-8 days. All growth factors and hypoxanthine promoted growth individually and in combination (FIG. 1 and Table 1).

TABLE 1
HF explant growth assays for individual growth factors, hypoxanthine and
growth cocktails GF7 and GF10. Each experimental point represents the
mean ± SEM of 6-8 independent experiments where an increase in
HF growth as a % of the untreated control is expressed.

| | Increase in | |
|---|---|---|
| | growth (% | |
| | control) | |
| | Mean | SEM |
| IGF1 | 39.30636 | 5.669739 |
| FGF2 | 146.474 | 17.09271 |
| FGF10 | 48.55491 | 9.449378 |
| PDGF | 69.55684 | 18.84979 |
| Wnt | 104.817 | 22.5991 |
| Nog | 51.44509 | 11.85743 |
| Eph | 57.61079 | 11.55394 |
| SHH | 125.8189 | 26.08114 |
| BMP-6 | 46.43545 | 5.533585 |
| Hypx | 104.1618 | 13.27246 |
| GF7 | 169.3642 | 19.07017 |
| GF10 | 158.1338 | 30.3139 |

### Cells

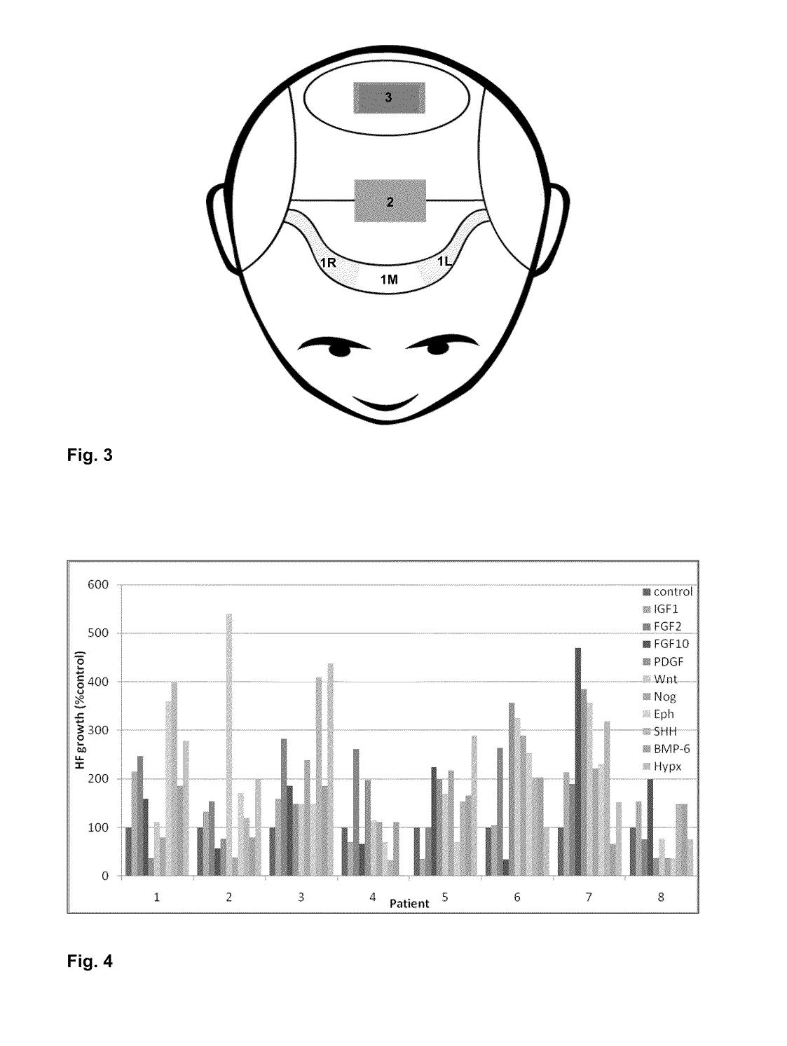

Cell viability assays were performed for dermal papilla (DP) cells. Dermal papilla cells were isolated for hair follicles and cultures under standard cell culture conditions. Cells were treated with varying concentrations of the GF10 cocktail (all nine growth factors and hypoxanthine) for six days. Viability of the cells was analysed following a 3 hour incubation with MTS cell viability reagents. The GF10 cocktail increased DP cell viability compared to the non-treatment control at all tested concentrations (FIG. 2). Each concentration was tested in triplicate. Error bars correspond to the standard error of the mean.

### Example 2

### Assessing the Effects of the Growth Promoting Composition in Patients in Different Bald Areas

The GF10 cocktail (see Example 1) was applied in 4 different bald areas of the scalp (FIG. 3), as a topical application for 4-6 months in four patients. Total hair densities (vellus, miniaturized and terminal hair) were measured before and after treatment with a Folliscope system and increases in hair densities determined. Differences in response were observed, where bald areas IL+IR responded best (45% increase), followed by areas 2 and 3 (22.755 and 26.25% respectively); the least effect was observed for area IM (17.25%) (Table 2), suggesting that the effect of growth factors are bald area specific.

TABLE 2
Increase in hair follicle densities in patients in different hair loss areas
after 5-6 months of topical application of GF10. Each point represents
the average of 4 individual patients.

| | Average increase in hair follicle |
|---|---|
| | density per cm² after 5-6 months of |
| Hair loss/Bald area | topical GF10 application |
| 1M | 17.25 ± 5.85% |
| 1L + 1R | 45 ± 10.72% |
| 2 | 22.75 ± 7.53% |
| 3 | 26.25 ± 8.25% |

### Example 3

### Assessing Effect of a Growth Promoting Composition on Hair Growth in Different Patients

The GF10 cocktail (see Example 1) was applied as a topical application on four different bald areas twice-a-day for a 4-6 month period. Total hair densities (vellus, miniaturized and terminal hair) were measured before and after treatment with a Folliscope system. Average cumulative increases in hair densities determined over all 4 bald areas for each patient. Differences in response were observed for the four patients, where patient 1 showed the best response (162% increase) followed by patients 3 and 4 with 69% and 71% increases respectively while the decrease (−4.5%) in hair growth observed for patient 2 suggests variability among individual patients (Table 3).

TABLE 3
Changes in hair follicle densities (positive, increase; negative, decrease) of
4 patients in response to GF10 application for 4-6 months. Total HF
densities were measured before and after treatment with trichometry
(Folliscope).

| | Increase in hair follicle density per |
|---|---|
| | cm² after 4-6 months of topical |
| Patient | GF10 application |
| 1 | 162% |
| 2 | −4.5% |
| 3 | 69% |
| 4 | 71% |

### Example 4

### Hair Follicle Growth Factor Profile for Different Patients

Hair follicles were extracted from individual patients and growth factor profiles were generated per individual patient using in vitro HF explant assays; results expressed as % increase in HF growth compared to untreated control as in Example 1. The results in FIG. 4 demonstrate the significant variability in GF profiles among individual patients.

### Example 5

### Hair Growth Before and after Treatment with Growth Cocktail GF10

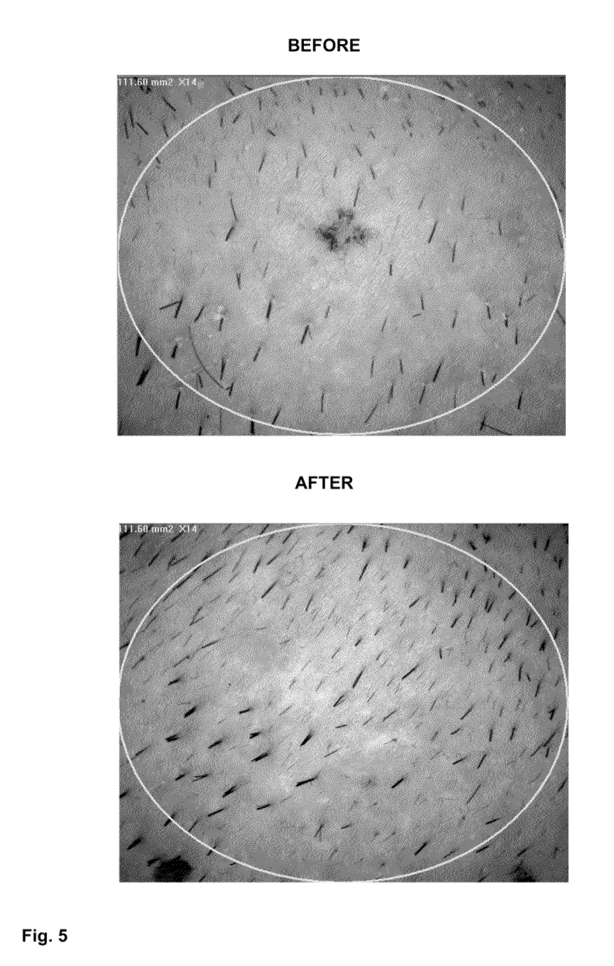

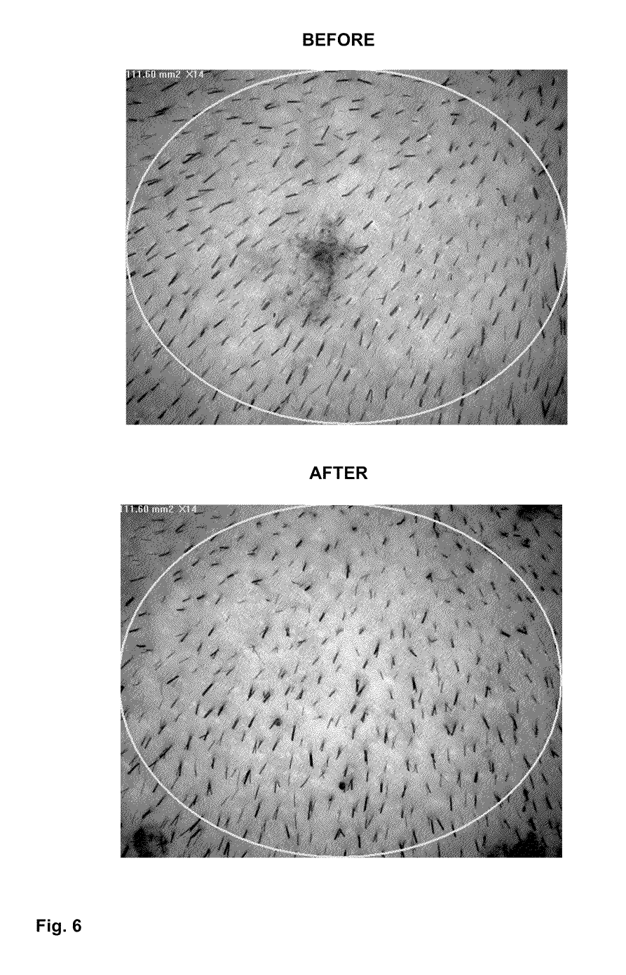

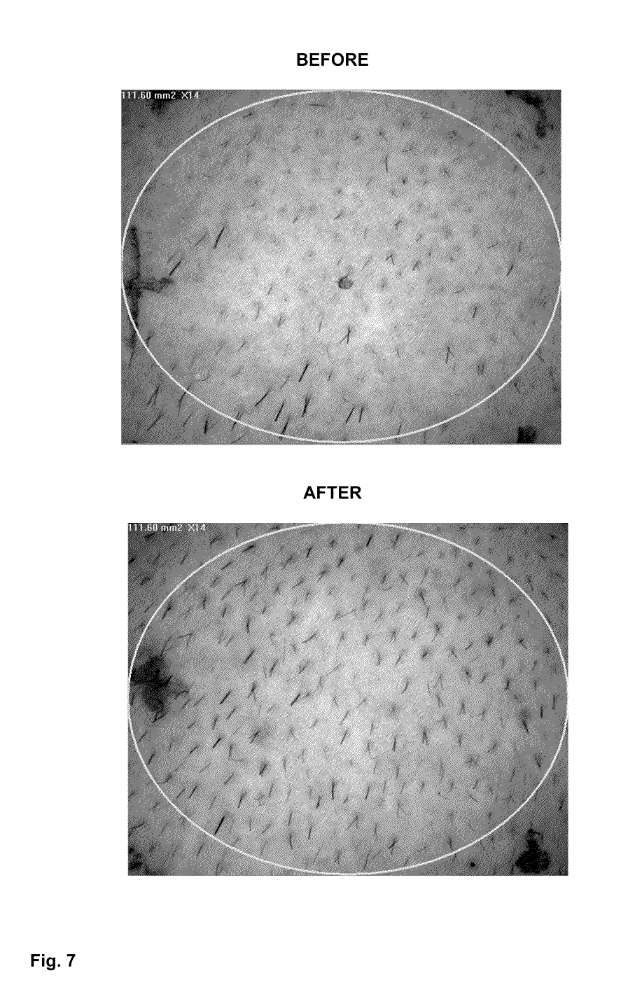

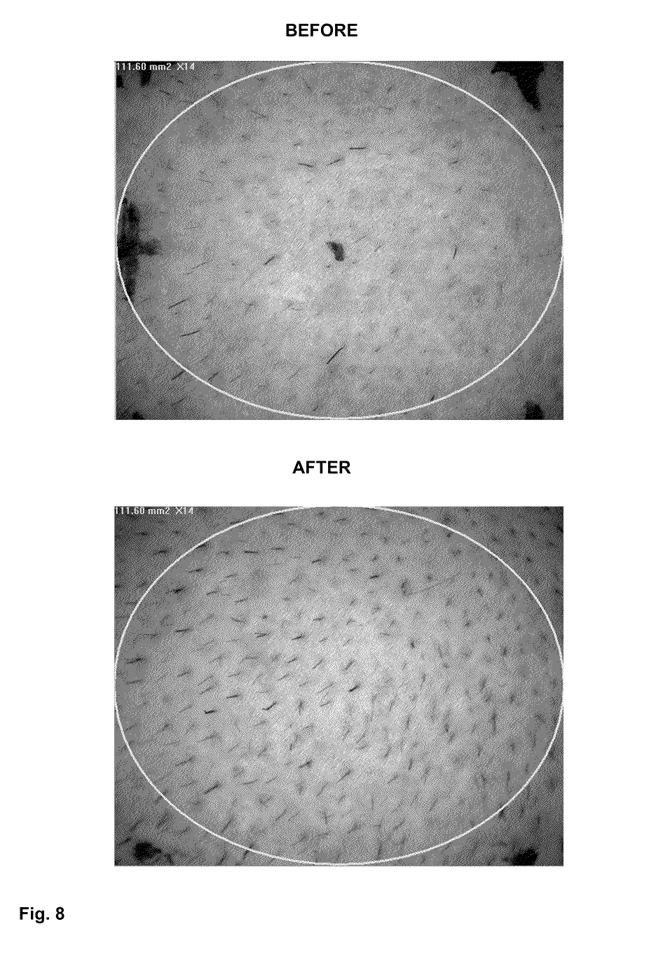

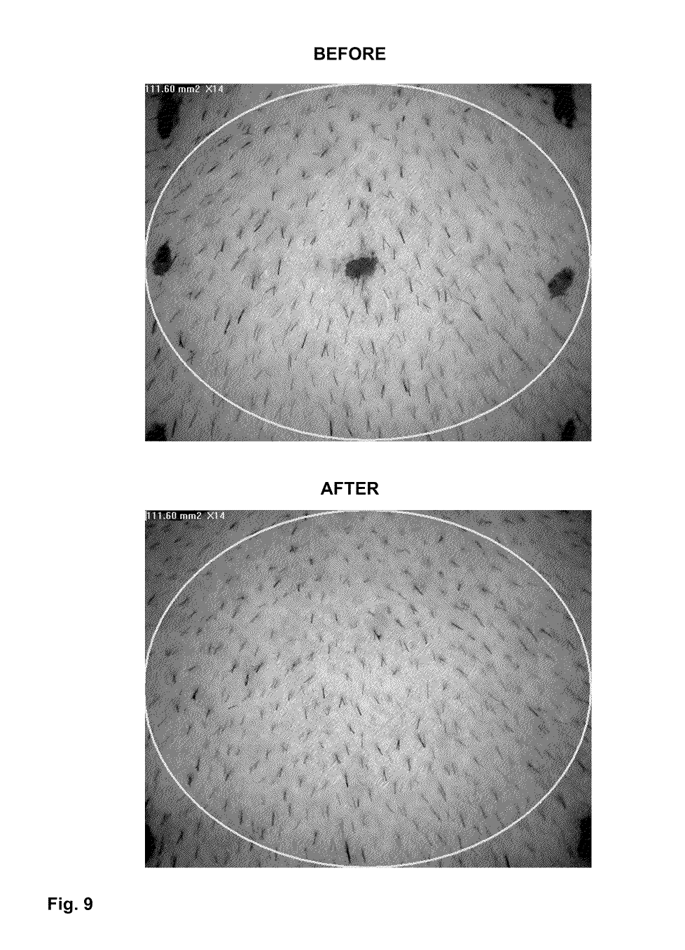

Patient 1 was treated for 13 weeks with a twice daily topical application of growth cocktail GF10 containing 20 ng/ml of each growth factor and 2 nmol/ml hypoxanthine (2 ml total per day) in bald areas 1 and 3 (see FIG. 3 for definition of bald areas). Patients 2 and 3 were treated for 16 weeks and 23 weeks, respectively, Patient 2 was treated in bald areas 2 and 3 and Patient 3 was treated in bald area 1. FIGS. 5-9 show photographs of hair growth before and after treatment. FIG. 5 shows bald area 1 of patient 1 before and after treatment. FIG. 6 shows bald area 3 of patient 1 before and after treatment. FIG. 7 shows bald area 2 of patient 2 before and after treatment. FIG. 8 shows bald area 3 of patient 2 before and after treatment. FIG. 9 shows bald area 1 of patient 3 before and after treatment.

### Example 6

### Hair Growth Before and after Treatment with Growth Cocktail GF10

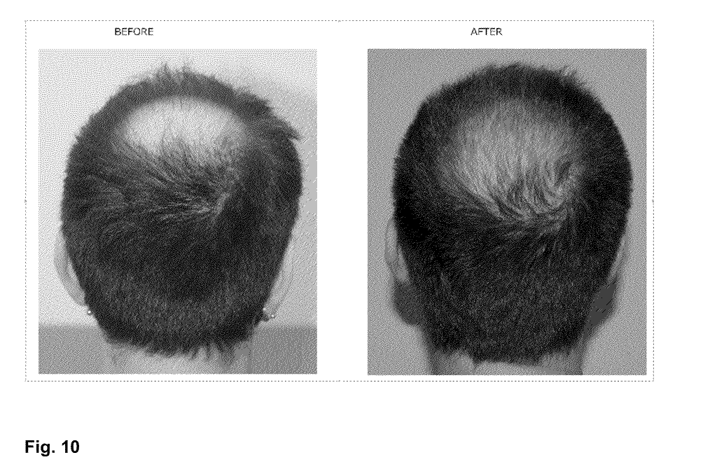

One ml of the GF10 composition (20 ng/ml of each growth factor and 2 nmol/ml hypoxanthine) was topically applied to the scalp of a subject twice daily for three months. FIG. 10 is a photograph showing hair growth before and after treatment.

### REFERENCES

Andl, T., S. T. Reddy, T. Gaddapara, and S. E. Millar. 2002. WNT signals are required for the initiation of hair follicle development. Dev. Cell 2:643-653.
Arnold, I. and F. M. Watt. 2001. c-Myc activation in transgenic mouse epidermis results in mobilization of stem cells and differentiation of their progeny. Curr. Biol. 11:558-568.
Botchkarev, V. A., N. V. Botchkareva, P. Welker, M. Metz, G. R. Lewin, A. Subramaniam, S. Bulfone-Paus, E. Hagen, A. Braun, M. Lommatzsch, H. Renz, and A. R. Paus. 1999. A new role for neurotrophins: involvement of brain-derived neurotrophic factor and neurotrophin-4 in hair cycle control. FASEB J 13:395-410.
Chiang, C., R. Z. Swan, M. Grachtchouk, M. Bolinger, Y. Litingtung, E. K. Robertson, M. K. Cooper, W. Gaffield, H. Westphal, P. A. Beachy, and A. A. Dlugosz. 1999. Essential role for Sonic hedgehog during hair follicle morphogenesis. Dev. Biol. 205:1-9.
Fuchs, E. 2007. Scratching the surface of skin development. Nature 445:834-842.
Holder, N. and R. Klein. 1999. Eph receptors and ephrins: effectors of morphogenesis. Development 126:2033-2044.
Jang, J. H. 2005. Stimulation of human hair growth by the recombinant human keratinocyte growth factor-2 (KGF-2). Biotechnol. Lett. 27:749-752.
Karlsson, L., C. Bondjers, and C. Betsholtz. 1999. Roles for PDGF-A and sonic hedgehog in development of mesenchymal components of the hair follicle. Development 126:2611-2621.
Katsuoka, K., H. Schell, O. P. Hornstein, and B. Wessel. 1987. Epidermal growth factor and fibroblast growth factor accelerate proliferation of human hair bulb papilla cells and root sheath fibroblasts cultured in vitro. Br. J. Dermatol. 116:464-465.
Kishimoto, J., R. E. Burgeson, and B. A. Morgan. 2000. Wnt signaling maintains the hair-inducing activity of the dermal papilla. Genes Dev. 14:1181-1185.
Morrell N T, et al. Liposomal packaging generates Wnt protein with in vivo biological activity. PLoS One. 2008 Aug. 13; 3(8):e2930.
Noramly, S. and B. A. Morgan. 1998. BMPs mediate lateral inhibition at successive stages in feather tract development. Development 125:3775-3787.
O'Shaughnessy, R. F., A. M. Christiano, and C. A. Jahoda. 2004. The role of BMP signalling in the control of ID3 expression in the hair follicle. Exp. Dermatol. 13:621-629.
Ozeki, M. and Y. Tabata. 2003. In vivo promoted growth of mice hair follicles by the controlled release of growth factors. Biomaterials 24:2387-2394.
Philpott, M. P., D. A. Sanders, and T. Kealey. 1994. Effects of insulin and insulin-like growth factors on cultured human hair follicles: IGF-I at physiologic concentrations is an important regulator of hair follicle growth in vitro. J. Invest Dermatol. 102:857-861.
Rendl, M., L. Lewis, and E. Fuchs. 2005. Molecular dissection of mesenchymal-epithelial interactions in the hair follicle. PLoS. Biol. 3:e331.
Rendl, M., L. Polak, and E. Fuchs. 2008. BMP signaling in dermal papilla cells is required for their hair follicle-inductive properties. Genes & Development 22, (4) (February 15): 543-57.
St Jacques, B., H. R. Dassule, I. Karavanova, V. A. Botchkarev, J. Li, P. S. Danielian, J. A. McMahon, P. M. Lewis, R. Paus, and A. P. McMahon. 1998. Sonic hedgehog signaling is essential for hair development. Curr. Biol. 8:1058-1068.
Waikel, R. L., Y. Kawachi, P. A. Waikel, X. J. Wang, and D. R. Roop. 2001. Deregulated expression of c-Myc depletes epidermal stem cells. Nat. Genet. 28:165-168.
Zanet, J., S. Pibre, C. Jacquet, A. Ramirez, I. M. de Alboran, and A. Gandarillas. 2005. Endogenous Myc controls mammalian epidermal cell size, hyperproliferation, endoreplication and stem cell amplification. J Cell Sci 118:1693-1704.

We claim:

1. A composition comprising at least four hair growth agents selected from the group consisting of: IGF-1, FGF-2, FGF-10, PDGF-AA, Wnt-3a, noggin, ephrin-A3, SHH, BMP-6 and hypoxanthine.

2. The composition of claim 1, wherein the composition comprises IGF-1, FGF-2, PDGF-AA, Wnt-3a, noggin, BMP-6 and hypoxanthine.

3. The composition of claim 1, wherein the composition comprises 10-30 ng/ml IGF-1, 10-30 ng/ml FGF-2, 10-30 ng/ml PDGF-AA, 10-30 ng/ml Wnt-3a, 10-30 ng/ml noggin, 10-30 ng/ml BMP-6 and 1-3 μM hypoxanthine.

4. The composition of claim 1, wherein the composition comprises 15-25 ng/ml IGF-1, 15-25 ng/ml FGF-2, 15-25 ng/ml PDGF-AA, 15-25 ng/ml Wnt-3a, 15-25 ng/ml noggin, 15-25 ng/ml BMP-6 and 1.5-2.5 μM hypoxanthine.

5. The composition of claim 1, wherein the composition comprises IGF-1, FGF-2, FGF-10, PDGF-AA, Wnt-3a, noggin, ephrin-A3, SHH, BMP-6 and hypoxanthine.

6. The composition of claim 1, wherein the composition comprises 10-30 ng/ml IGF-1, 10-30 ng/ml FGF-2, 10-30 ng/ml FGF-10, 10-30 ng/ml PDGF-AA, 10-30 ng/ml Wnt-3a, 10-30 ng/ml noggin, 10-30 ng/ml ephrin-A3, 10-30 ng/ml SHH, 10-30 ng/ml BMP-6, and 1-3 μM hypoxanthine.

7. The composition of claim 1, wherein the composition comprises 15-25 ng/ml IGF-1, 15-25 ng/ml FGF-2, 15-25 ng/ml FGF-10, 15-25 ng/ml PDGF-AA, 15-25 ng/ml Wnt-3a, 15-25 ng/ml noggin, 15-25 ng/ml ephrin-A3, 15-25 ng/ml SHH, 15-25 ng/ml BMP-6, and 1.5-2.5 μM hypoxanthine.

8. A method of increasing hair growth and/or decreasing hair loss in a subject in need thereof, wherein the method comprises administering the composition of claim 1 to the subject.

9. The method of claim 8, wherein the composition is administered topically.

10. The method of claim 8, wherein the composition is administered twice daily.

11. The method of claim 8, wherein the composition is administered in an amount of 0.5-5 ml.

12. The method of claim 8, wherein the composition is administered in an amount of 1-3 ml.

13. The method of claim 8, wherein the composition is administered to the scalp of the subject.

14. The method of claim 13, wherein the composition is administered to a thinning hair patch or a bald patch on the scalp.

15. A method of increasing the viability of a hair follicle cell, wherein the method comprises contacting the hair follicle cell with the composition of claim 1.

16. The method of claim 15, wherein the hair follicle cell is a cell selected from the group consisting of: epidermal matrix cells, dermal papilla cells, dermal sheath cells and outer root sheath cells.

17. A method of preparing a composition useful increasing hair growth or decreasing hair loss, the method comprising:
providing a composition comprising at least four hair growth agents selected from the group consisting of: IGF-1, FGF-2, FGF-10, PDGF-AA, Wnt-3a, noggin, ephrin-A3, SHH, BMP-6 and hypoxanthine.

18. The method of claim 17, wherein the composition comprises IGF-1, FGF-2, PDGF-AA, Wnt-3a, noggin, BMP-6 and hypoxanthine.

19. The method of claim 17, wherein the composition comprises IGF-1, FGF-2, FGF-10, PDGF-AA, Wnt-3a, noggin, ephrin-A3, SHH, BMP-6 and hypoxanthine.

20. The method of claim 17, wherein the method further comprises administering the composition to a subject to increase hair growth or decrease hair loss on the subject.
